(19) United States
(12) Patent Application Publication
(10) Pub. No.: US 2024/0285366 A1
(43) Pub. Date: Aug. 29, 2024

(54) CREATION AND USE OF PANORAMIC VIEWS OF A SURGICAL SITE

(71) Applicant: Asensus Surgical US, Inc., Durham, NC (US)

(72) Inventor: Motti Frimer, ZICHRON YAAKOV (IL)

(21) Appl. No.: 18/499,189

(22) Filed: Oct. 31, 2023

Related U.S. Application Data
(60) Provisional application No. 63/420,833, filed on Oct. 31, 2022.

(51) Int. Cl. A61B 90/00

(52) U.S. Cl. CPC A61B 90/361; A61B 90/37; A61B 2090/364

(57) ABSTRACT

In a system and method for creating a panoramic view of a surgical procedure, a camera is positioned in a body cavity and captures real time images of a surgical site within the body cavity. The real time images are stitched together with non-real time images of the body cavity, that were captured during the surgical procedure prior to time t. The stitching of the real-time and non-real-time images creates a panoramic image that is displayed on a display.

### CROSS-REFERENCE TO RELATED APPLICATIONS

This application claims the benefit of co-pending US Provisional Application No. 63/420,833, filed Oct. 31, 2022.

### BACKGROUND OF THE INVENTION

During surgery, cameras are used to capture 2D and/or 3D images of the surgical site within the body cavity. The images are displayed on an image display that is observed by the practitioner as the surgery is performed. Because the cameras (typically the type commonly referred to as laparoscopes or endoscopes) capture a relatively small field of view at any given moment, the displayed images offer the surgeon a view of only a small area within the body cavity.

Acquiring measurement data from a surgical site can be useful to a surgeon or other practitioner. Co-pending and commonly owned U.S. application Ser. No. 17/099,761, entitled “Method and System for Providing Surgical Site Measurements” (which is attached at the Appendix) describes a system and method that use image processing of images of the endoscopic view to estimate or determine distance measurements between identified measurement points at the treatment site. The measurements may be straight line point to point measurements, or measurements that follow the 3D topography of the tissue positioned between the measurement points. Measurements may be taken between parts of the instruments disposed within the field of view of the camera, or between icons or tags graphically positioned by the user using an input device.

### BRIEF DESCRIPTION OF DRAWINGS

FIG. 1 schematically illustrates an embodiment of a system suitable for creating and using panoramic views of a surgical site.

FIG. 2A schematically depicts image data captured of a region of a body cavity in which a surgical site is located;

FIG. 2B schematically depicts a 3D topographic image generated using the image data represented in FIG. 2A;

FIG. 3A is similar to FIG. 2A, but shows image data captured of multiple regions stitched together to create a panoramic view of a larger area of the body cavity; schematically depicts image data captured of a region of a surgical site;

FIG. 3B is similar to FIG. 2B, but shows image data captured of multiple regions stitched together to create a topographic panoramic view of a larger area of the body cavity;

FIG. 4A shows an image captured by an endoscope positioned at a surgical site in body cavity and displayed in real time;

FIG. 4B shows the image shown in FIG. 4A as displayed in real time during a surgical procedure and as stitched together with non-real time images captured by the endoscope at a previous point in time during the same surgical procedure. An optional border shown in the image delineates the real-time image from the non-real-time image;

FIG. 5A shows an image captured by an endoscope positioned at a surgical site in body cavity and displayed in real time;

FIG. 5B shows the image shown in FIG. 5A as displayed in real time during a surgical procedure and as stitched together with non-real time images captured by the endoscope at a previous point in time during the same surgical procedure. In the real-time image, the surgical instruments positioned at the surgical site are digitally removed from the image and replaced with overlays marking only the outlines of the instrument.

FIG. 6 depicts a panoramic image generated during a surgical procedure, in which data marking tools used, tool modes, tool actions, surgical insights, and other data have been integrated.

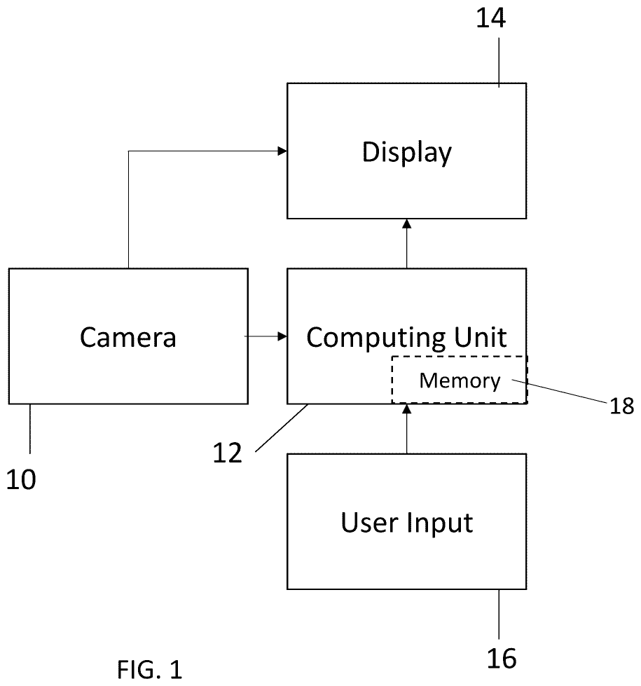

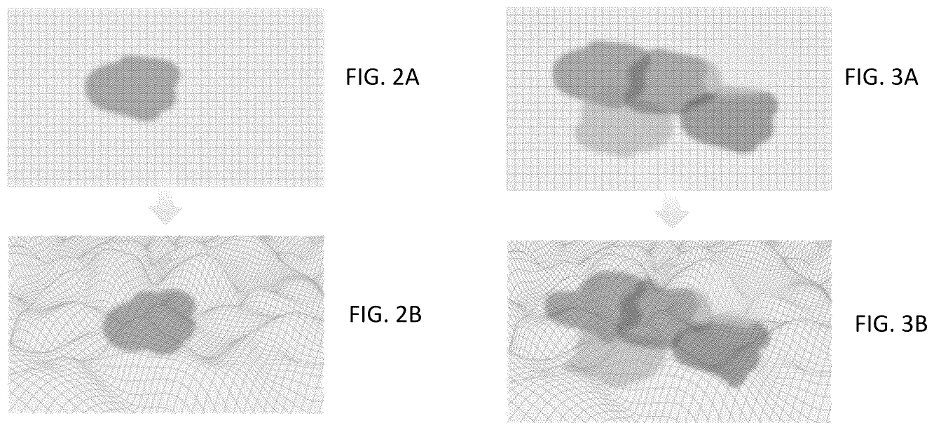

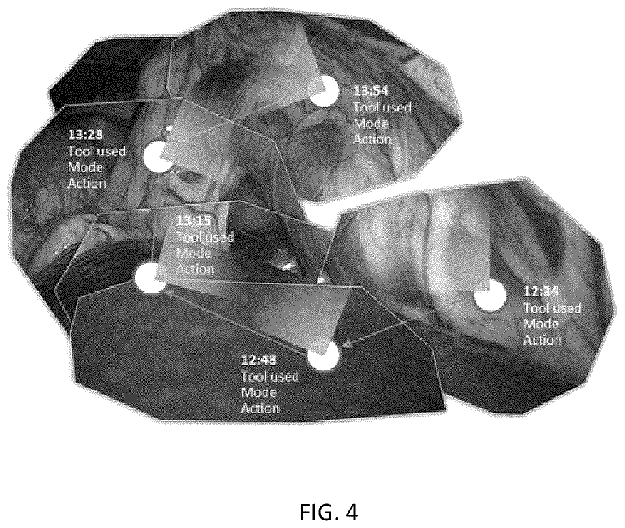

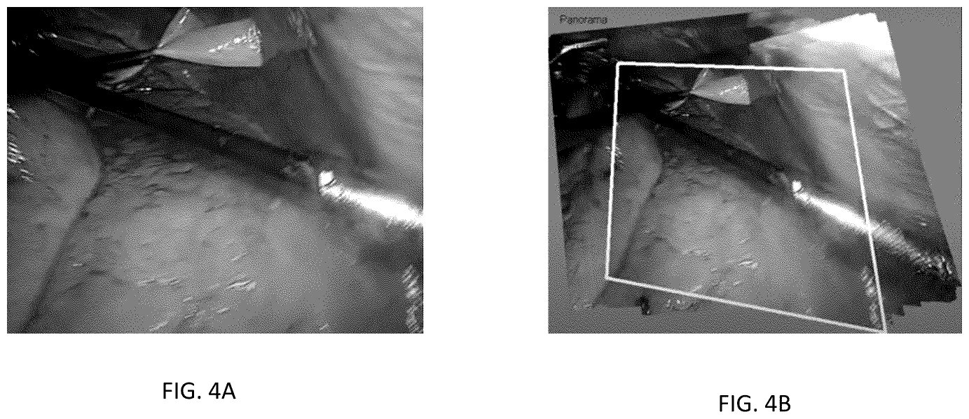

### DETAILED DESCRIPTION OF THE INVENTION

This application describes a system and method for use in conjunction with surgical instruments that are used to perform diagnostic or therapeutic tasks at a surgical site. In particular, the system can stitch together 3D and/or 2D images to create a panoramic view of the surgical site, which is displayed to the surgeon in 3D or 2D during surgery.

The system comprises a camera 10 positionable in a body cavity to capture real-time images of a surgical site within the body cavity, such as a laparoscopic/endoscopic camera. The system further has at least one computing unit 12 having at least one memory 18 storing instructions executable by the computing unit 12 to perform the functions described herein, including: receiving real-time images captured by the camera 10 at a time t, stitching the real time images with non-real-time images of the body cavity, said non-real-time images captured during the surgical procedure prior to time t, where said stitching creates a panoramic image, and displaying the panoramic image on a image display 14.

As the user maneuvers the camera within the body camera, the panoramic image is updated so as to display the most current image data available for each region of interest. The result is a bigger picture of the scene that helps the surgeon better see both the region in which the surgeon is currently working, as well as regions in which the surgeon was working or exploring earlier in the procedure. This provides the surgeon with a greater understanding of a larger portion of the scene.

As shown in the figures, where a 3D panorama is generated using 3D camera data, 3D mapping information may be generated from image data captured by the camera. This data may include 3D topographical data. Thus, over time, as the camera is moved within the body camera (either manually or using a robotic manipulator), image data corresponding to multiple areas within the surgical site are stitched together to form a topographical panoramic map.

Based on the 3D reconstruction output for each processed image frame, a topographic 3D model of the surgical scene is constructed and thus displayed, providing a 3D view of the scene. The user may give input to the system using a user input device 16, instructing the system to manipulate the displayed model, such as by rotating, expanding or contracting the model, or by changing the angle of view of the displayed panoramic image. The input may be given using a touch screen, microphone for voice commands, keyboard, wireless controller, mouse, foot pedal, eye tracking input as confirmed by some other input means (including any of those listed in this sentence), using switches, buttons, knobs etc. on a handle of a user input device of a surgical robotic system, or any other user interface. These manipulations may be performed during the surgical procedure, when the panoramic image is comprised of real-time images stitched with non-real time images. Moreover, as described below, such manipulations may similarly be performed during or after the surgical operation when the panoramic image is comprised entirely of non-real time images captured during the course of single surgical procedure.

The panoramic image may also be stored in memory by the system processor, so the user can replay the panoramic video, and optionally interact with it as will be further described below.

The system may perform certain modifications to the displayed image for the convenience of the user. For example, the system may use known computer vision techniques to detect the surgical instruments within the visual field, and to “erase” the surgical instruments from the portions of the panoramic display that display non-real-time images. This allows the user to see only the present location of the instruments on the display, and it prevents the panoramic view from being cluttered with multiple images of instruments in spots where they were positioned several minutes prior. As an alternative, rather than completely erasing the instruments, the images of the surgical instruments may be replaced with simple outlines of the instruments.

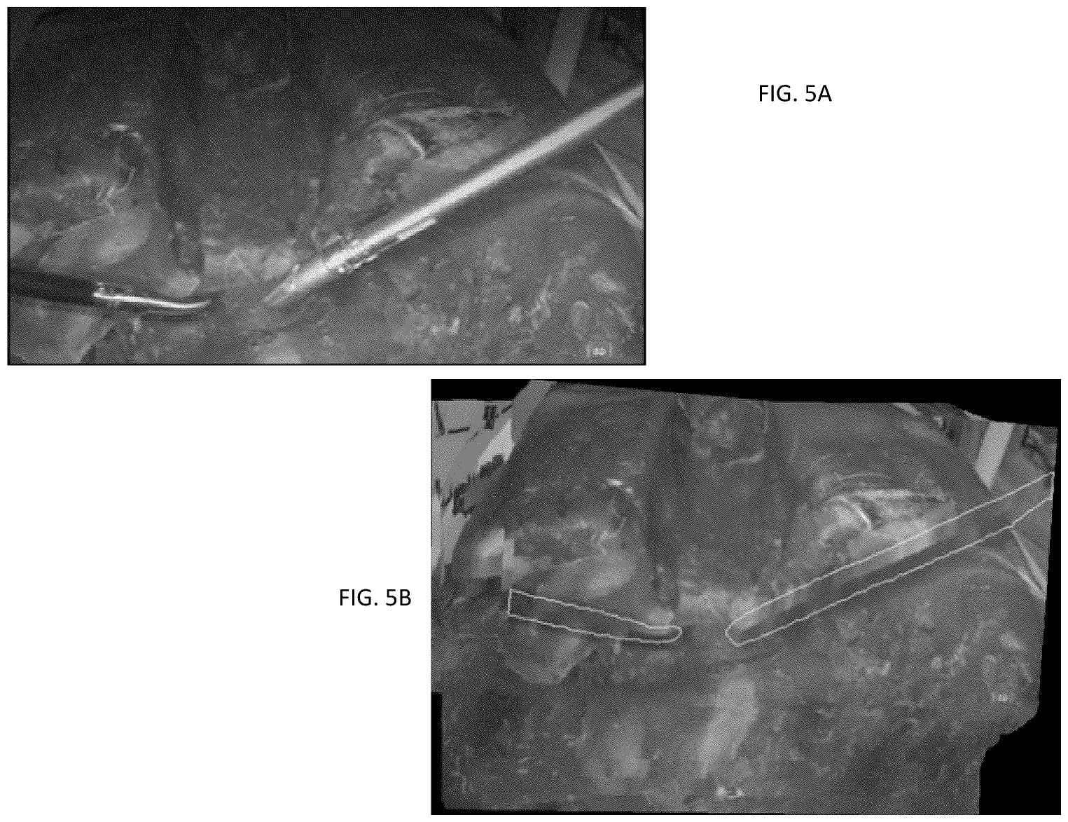

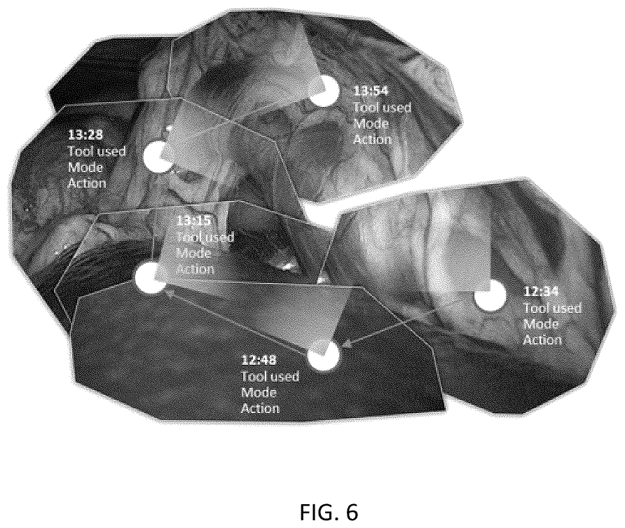

As one example, FIG. 5A shows an image captured by an endoscope positioned at a surgical site in body cavity and displayed in real time. In FIG. 5B, the real-time image shown in FIG. 5A is displayed in real time as stitched together with non-real time images captured by the endoscope at a previous point in time during the same surgical procedure. In the non-real time images, the surgical instruments positioned at the surgical site are completely erased. In the real-time image, the surgical instruments positioned at the surgical site are digitally removed from the image and replaced with overlays marking only the outlines of the instrument.

In alternative embodiments, some of the non-real-time images making up the displayed panoramic image may be shown without the surgical instruments erased, or with the surgical instruments replaced with outlines or other depictions of the surgical instruments (e.g. the outlines shown in FIG. 5B). One example of a context in which this might be desirable is when the user has moved the endoscope so some or all of the surgical instruments are no longer in the field of view captured by the endoscope. In that situation, it may be useful for the user to be able to see the instruments in the non-real-time images forming part of the panoramic image.

As another example, the panoramic image may be generated in a way that makes it easy for the user to know which portions of the panoramic image are relatively outdated. For example, the portions of the stitched imaged that resulted from images captured more than a pre-determined number of minutes prior (e.g., 3 minutes prior) might be displayed as slightly shaded or muted in their coloring, or they might be marked with a overlay of a border around them, or with another form or graphical or textual overlay that signals to the user that they have become aged non-real-time images. A user, upon seeing such shading or muted coloring, may then choose to maneuver the camera around the body cavity to capture new images of the various regions of the surgical site, causing the system to update those regions in the panoramic image and giving the user a refreshed view of the site. The system thus may be configured to track the amount of time that has lapsed since each non-real-time image was initially captured in real time. When the elapsed time for any given non-real-time image exceeds a predetermined duration, the user is alerted using such a visual alert.

Each portion of the panoramic image that is refreshed as described above will initially be a real-time image, then it will become a non-real-time image.

The recorded data may also include actions taken within the surgical field. For example, when measurements are taken as discussed in the prior application, data corresponding to the measurements may be recorded. This data may include a variety of elements, such as (i) the 3d locations, images or graphical depictions of the points between which the measurements were taken (e.g., graphical tags marking the points, or, where measurements are taken between points on the instrument, depictions of those points), (ii) the graphical depictions of the straight-line or contoured connectors between the measurement points, and (iii) graphical depictions of the measurements themselves may be captured in the data. Notations or graphical depictions highlighting other steps taken in the procedure (and the points on the tissue at which they were taken) may be also captured, such as application of sutures or staples to the tissue, sites at which energy is applied to the tissue or at which cuts made in the tissue, etc. Data reflecting the most frequent actions and/or locations of certain of the surgical tools within the site may also be recorded.

The panoramic image may be saved by the system in an associated memory, so the surgical team, surgical trainees, etc. may “replay” the surgical procedure. As the panoramic images are replayed, the user may re-visit measurements and other actions taken during the surgery and make take new measurements. For example, the user may choose to take measurements between different measurement points than were used during the actual surgery. The system may allow these subsequent measurements to be performed in a manner similar to what is described in U.S. application Ser. No. 17/099,761, with the user identifies measurement points to the system using a graphical user interface. If graphical tags were placed by the user during the surgery, those tags may be viewed during the replay of the procedure. During the surgical procedure, the surgeon might assign names to the tags (e.g. specifying the anatomical feature or structure identified by the tag, or specifying a step in the procedure marked in time by the tag), and during the replay of the panoramic video the user may be given a list of the tags and the opportunity to select a tag to be displayed. The user can view the video from various angles.

It can thus be envisioned that a surgical simulator may be created, which allows a user to perform training activities using one of a variety of panoramic videos that have been generated in actual surgeries. This enhances surgeon training by providing simulation using actual 3D topography instead of graphically simulated topography. The panoramic videos available on such a simulator may be organized to allow a user to input desired surgical type and patient characteristics. For example, the surgeon might specify an interest in simulating a procedure on a male patient with a body mass index of 44 and some other characteristic, such as a fatty liver. The system will select the most relevant panoramic video in its database to use for the simulation or offer the surgeon a list of relevant ones to choose from.

Using the 3D model of the surgical scene the surgeon will have the option to apply different measurements applications such as measuring the distance between two points, areas measurement, and volume measurement. In addition, the surgeon would have the option to evaluate different types of mesh on the surface of the 3D model and evaluate the level of suitability of a mesh to the required action (e.g find a suitable mesh to cover hernia defect). The size of the mesh can be predefined or according to the measured area depending on the surgeon's need. Features described in co-pending U.S. application Ser. No. 17/035,534, entitled Method and System for Providing Real Time Surgical Site Measurements may be used in this context.

Other features that may be used in conjunction with the disclosed concepts include:
Applying digital markers, such as graphical or textual overlays, on the panorama—using the 3D model of the surgical scene the surgeon will have the option to add markers on the 3D model.

Camera location and orientation—as part of the panoramic view in the case of the 3D panorama the surgeon would get an indication of the location and orientation of the camera in relation to the 3D model of the surgical scene in order to improve his understanding of the 3D model and improve his localization. The camera location during the entire operation will be saved and could be used during post ops analysis.

Presenting current camera location and saved landmarks-in the case of a 2D panoramic view, the current view will be highlighted and marked. Additionally, any saved landmark/location (which the surgeon has the option to get back to or not) will be presented on the panoramic view.

Giving an indication about tools or tags outside of the FOV-using the data from the panorama the system will be able to give the surgeon indication about tools, objects, and markers (that were placed by the surgeon) in case they are getting out of the field of view. The information will be based only on data that was acquired from the image.

Using blending techniques to overcome overlay and stitching artifacts—integrating an image to a current 2D/3D model, may hold different types of artifacts in the seam-line border and in the integrated model. These artifacts are caused by a difference in lumination, contrast, registration and more. Different types of blending techniques, such as alpha channel blending, feathering, Laplacian pyramid and voxel hashing, are used to minimize the visibility of seams between image to model.

All prior patents and patent applications referenced herein are incorporated herein by reference.

1. A system for creating a panoramic view of a surgical procedure, the system comprising;
a camera positionable in a body cavity for capturing real time images of a surgical site within a body cavity;
a display for displaying the real time images in real times;
at least one computing unit and at least one memory storing instructions executable by the at least one computing unit to
receive the real time images captured by the camera at time t during a surgical procedure;
stitch the real time images with non-real time images of the body cavity, said non-real time images captured during the surgical procedure prior to time t, where said stitching creates a panoramic image; and
display the panoramic image on the display.

2. The system of claim 1, wherein the instructions are executable by the at least one computing unit to repeat the receiving, stitching and displaying steps multiple times during the course of the surgical procedure.

3. The system of claim 1, wherein the instructions are executable by the at least one computing unit to alert the user if a displayed non-real time image is an aged non-real time image.

4. The system of claim 3, wherein a non-real time image is an aged non-real time image if a period of time between initial capture of said non-real time image and a present time exceeds a predetermined duration.

5. The system of claim 4, wherein the instructions are executable by the at least one computing unit to determine if the displayed non-real time image is an aged non-real time image.

6. The system of claim 3, where the alert comprises altering the color of a non-real time image in said panoramic image after said non-real time image is determined to be an aged non real-time image.
